SEQUENCE LISTING (6 sequences)

SEQ ID NO: 1 - DNA, 20 bp, synthetic construct
  source 1..20: mol_type = other DNA
agcttcaggcaggcagtatc

SEQ ID NO: 2 - DNA, 20 bp, synthetic construct
  source 1..20: mol_type = other DNA
aaggtccacgggaaagacac

SEQ ID NO: 3 - DNA, 21 bp, synthetic construct
  source 1..21: mol_type = other DNA
aaatggcctccctctcatcag

SEQ ID NO: 4 - DNA, 26 bp, synthetic construct
  source 1..26: mol_type = other DNA
gtcactcgaattttgagaagatgatc

SEQ ID NO: 5 - DNA, 21 bp, synthetic construct
  source 1..21: mol_type = other DNA
cgtgcgtgacatcaaagagaa

SEQ ID NO: 6 - DNA, 20 bp, synthetic construct
  source 1..20: mol_type = other DNA
tggatgccacaggattccat

(19) United States
(12) Patent Application Publication
(10) Pub. No.: US 2025/0134894 A1
(43) Pub. Date: May 1, 2025

(54) COMPOSITIONS FOR PREVENTING AND TREATING NEURODEGENERATIVE DISEASES

(71) Applicant: ARIBIO CO., LTD., Gyeonggi-do 13535 (KR)

(72) Inventors: Jai Jun Choung, Gyeonggi-do (KR); Byungwoo Kang, Gyeongsangnam-do (KR)

(21) Appl. No.: 19/003,405

(22) Filed: Dec. 27, 2024

Related U.S. Application Data
(63) Continuation of application No. PCT/IB2023/056712, filed on Jun. 28, 2023.
(60) Provisional application No. 63/367,282, filed on Jun. 29, 2022.

(51) Int. Cl. A61K 31/519; A61K 31/13; A61P 25/28

(52) U.S. Cl. CPC A61K 31/519; A61K 31/13; A61P 25/28

(57) ABSTRACT

The present invention provides a composition for preventing or treating a neurodegenerative disease containing a phosphodiesterase 5 inhibitor (PDE5 inhibitor) and an N-methyl-D-aspartate-receptor (NMDA-receptor) antagonist and a method using thereof, wherein the PDE5 inhibitor is mirodenafil, sildenafil, vardenafil, tadalafil, udenafil, dasantafil, avanafil, pharmaceutically acceptable salts, solvates, hydrates, or a mixture thereof; and the NMDA-receptor antagonist is selected from among memantine, amantadine, ketamine, traxoprodil, lanicemine, rislenemdaz, pethidine, levorphanol, methadone, dextropropoxyphene, tramadol, ketobemidone, dextromethorphan (DXM), phencyclidine (PCP), and methoxetamine (MXE), pharmaceutically acceptable salts, solvates, hydrates and a mixture thereof; and the neurodegenerative disease is dementia, Parkinson's disease (PD), Dementia with Lewy body (DLB), Alzheimer's disease (AD), Huntington's disease (HD), Multiple sclerosis (MS), Vascular Dementia (VaD), Amyotrophic Lateral Sclerosis (ALS), frontotemporal dementia, or a mixed etiologies thereof.

### CROSS-REFERENCE TO RELATED APPLICATIONS

This application claims the benefit of priority from U.S. Provisional Application Ser. No. 63/367,282, filed Jun. 29, 2022, the contents of which are incorporated herein by reference.

### FIELD

The present invention relates to a composition containing a phosphodiesterase 5 inhibitor (PDE5 inhibitor) and an N-methyl-D-aspartate-receptor (NMDA-receptor) antagonist for preventing or treating neurodegenerative diseases and a method using thereof.

### SEQUENCE LISTING

This application incorporates by reference in its entirety the Sequence Listing XML file entitled “04334900121_SequenceListing.xml (7 KB)”, which was created on Jun. 23, 2023, and filed electronically herewith.

### BACKGROUND

Recently, patients with degenerative neurological disorders have been increased rapidly. In the treatment of degenerative neurological disorder, the most important step is the prevention. However, the cause of the disease has not been clearly understood yet and thus a treatment method is still needed to be studied. The common pathological phenomenon of degenerative neurological disorders is the death of central nervous system cells. Unlike other organ cells, central nervous system cells are almost impossible to regenerate after cell-death, resulting in permanent loss of function. Thus, the methods for the treatment of such brain diseases developed so far are mainly focused on the prevention of nerve cell death.

Neurodegeneration, as a collective term, involves the progressive loss of structure or function of neurons, including death of neurons in various areas of the brain. Neurodegenerative diseases including Parkinson's disease (PD), Alzheimer's disease (AD), Huntington's disease (HD) and Multiple sclerosis (MS) are emerging as a serious challenge to the ageing population. Potential causes for neurodegeneration or neuronal cell death are oxidative stress, aggregation of toxic proteins such as beta-amyloid and chronic inflammation in the Central Nervous System (CNS).

Recent studies on Alzheimer's disease and Parkinson's disease, for example, provide that inflammatory reaction in the brain is a major cause of neuronal death. The increase of inflammation mediators and reactive oxygen has been confirmed in the cerebrospinal fluid of brain disease patients. Also, numbers of active microglial cells are observed in the area of brain damage, indicating brain inflammation is a major cause of Parkinson's disease. Inhibition of brain inflammation by neuroglial cells has become a target of treating degenerative neurological disorder. However, therapeutic agents that have been developed so far are only effective in regulating the symptoms of the disease but are not effective in treating degenerative neurological disorder itself.

Therefore, it is required to develop a preventive and therapeutic agent for degenerative neurological disorder based on the totally different concept from the conventional ones. For example, dementia is an acquired brain disease with multifaceted pathogenesis caused by various genetic and environmental risk factors and refers to a clinical disease that causes multiple cognitive deficits. The most common cause of dementia is AD which occurs mainly in elderlies and contribute to more than 60% of dementia.

Studies implicate inflammation in neurodegenerative diseases like Parkinson's disease, Alzheimer's disease, Huntington's disease, etc. Contrary to the traditionally held belief that the brain is an immune-privileged site due to the presence of the Blood-Brain Barrier (BBB), recent studies have established that the brain is fully capable of mustering an immune response. Inflammation in the brain does not involve the peripheral immune system and does not involve antibodies or T-Cells. The immune reaction in the brain depends on the synthesis of inflammatory components by Glial cells especially the resident phagocytes, which in the case of the brain, are the microglia.

Within the brain, glial cells play a critical role in maintaining a microenvironment of homeostasis that promotes neuronal survival. Microglia mediate innate immune responses to invading pathogens by secreting a myriad of factors that include, cytokines, chemokines, prostaglandins, reactive oxygen and nitrogen species, and growth factors. Therefore, pro- and anti-inflammatory responses must be tightly regulated to prevent the potential detrimental effects of prolonged inflammation-induced oxidative stress on vulnerable neuronal populations.

In the normal adult brain, microglial cells are usually in the resting state. When activated, these cells are known to release various types of pro-inflammatory molecules such as Nitric Oxide (NO) and cytokines which cause damage and cell death in the surrounding neurons. For example, activated microglia, accumulation of cytokines as well as nuclear factor kappa B (NF-.kappa.B) pathway activation has been found to contribute to the progression of neurodegenerative diseases.

On the other hand, research on amyloid beta protein (Aβ), which is known to be a common cause of hereditary and sporadic Alzheimer's disease, reported that even in normal people, Aβ is produced in small amounts in various parts of the body. In normal people, Aβ is rapidly degraded after being produced and does not accumulate in the body, but in the case of patients with Alzheimer's disease, Aβ is produced in an abnormally large amount and is accumulated in tissues without being degraded, resulting in the generation of senile plaques or excessive accumulation in places such as the hippocampus or cerebral cortex, which play an important role in memory and learning. The accumulated Aβ triggers an inflammatory response in surrounding cells. As a result, nerve cells become damaged and even the neural networks for maintaining the normal function of the brain end up being damaged. Furthermore, the accumulated Aβ produces a great deal of reactive oxygen that activates the signaling system that kills nerve cells.

Aβ is a part of amyloid precursor protein cleaved by β-secretase. There are various forms of Aβ depending on the number of amino acids constituting it. In the case of patients with Alzheimer's disease, the proportion of Aβ composed of 40 or 42 amino acids increases rapidly. There are many reports that Aβ induces neuronal cell death when treated with nerve cells cultured in vitro and that the mechanism of cell death is similar to the type of apoptosis seen in patients with Alzheimer's disease. Damage to nerve cells by Aβ1-42 or Aβ1-43 protein has been identified as one of the important causes of Alzheimer-type disease, and Aβ25-35 is known to be an important toxic fragment of Aβ1-42 or 43 that causes nerve cell damage.

The most common drugs currently approved by the FDA and used to treat dementia include acetylcholinesterase inhibitors (AchEIs) and NMDA receptor antagonists, and various other drugs, such as antioxidants, nonsteroidal anti-inflammatory drugs (NSAID), anti-inflammatory agents, statins, and hormones, are used in combination therewith. However, these drugs are only used to relieve and delay symptoms and improve cognitive function, and currently, a fundamental treatment for dementia is still in need.

Due to the decrease or loss of nerve cell function, degenerative neurological diseases including dementia show abnormalities in a wide variety of functions including all functions of the body that can be felt, such as the motor control function, cognitive function, perceptive function, and sensory function of the human body, as well as the autonomic nervous function, which is self-regulated in a state that the human body is not aware of.

Because the cause of the neurodegenerative diseases is not completely understood, a fundamental treatment is still at challenge. The commercially available drugs can only relieve symptoms in some diseases, but fail to fundamentally change the progression of the disease. The serious side effects of the drug emerge after treatment further limits the improvement of symptoms in these patients. Therefore, the options for treating neurodegenerative diseases or disorders including dementia are still limited.

### SUMMARY

The present invention provides a composition and a method for treating a neurodegenerative disease by reducing the neuroinflammation especially in CNS system and/or by reducing the expression of a toxic protein such as beta-amyloid (Ab),
wherein,
the composition comprising a PDE-5 inhibitor and an NMDA-receptor antagonist;
the PDE-5 inhibitor is selected from among mirodenafil, sildenafil, vardenafil, tadalafil, udenafil, dasantafil, avanafil; and pharmaceutically acceptable salts, solvates, hydrates, or a mixture thereof;
the NMDA-receptor antagonist is selected from among memantine, amantadine, ketamine, traxoprodil, lanicemine, rislenemdaz, pethidine, levorphanol, methadone, dextropropoxyphene, tramadol, ketobemidone, dextromethorphan (DXM), phencyclidine (PCP), and methoxetamine (MXE), pharmaceutically acceptable salts, solvates, hydrates or a mixture thereof;
the composition inhibits proinflammatory cytokines such as IL1b, IL-6, or TNFa;
the composition inhibits the growth and differentiation of nerve cells and degenerating learning and memory, to induce a decrease in intracellular Aβ, thereby increasing the protection of nerve cells and synaptic plasticity; and
the neurodegenerative disease is selected from among dementia, Parkinson's disease (PD), Dementia with Lewy body (DLB), Alzheimer's disease (AD), Huntington's disease (HD), Multiple sclerosis (MS), Vascular Dementia (VaD), Amyotrophic Lateral Sclerosis (ALS), frontotemporal dementia, or a mixed etiologies thereof.

### BRIEF DESCRIPTION OF THE DRAWINGS

FIG. 1 and FIG. 2 show that compositions containing mirodenafil (AR1001) and memantine provide synergistic effects on IL1β inhibition, for example, at the ratio of mirodenafil:memantine at 10:1, 5:1, 2:1, 1:1, 1:2, or 1:5.

FIG. 3 and FIG. 4 show that compositions containing mirodenafil and memantine provide synergistic effects on TNFα inhibition, for example, at the ratio of mirodenafil:memantine at 10:1, 5:1, 2:1, 1:1, 1:2, 1:5, or 1:10.

FIG. 5 and FIG. 6 show that compositions containing mirodenafil (AR1001) and memantine provide synergistic effects on reducing Aβ42 accumulation at the ratio of, for example, mirodenafil:memantine at 5:1, 1:1, or 1:5.

### DETAILED DESCRIPTION

The present invention provides a composition and a method for treating a neurodegenerative disease by reducing the neuroinflammation especially in CNS system and/or by reducing the expression of a toxic protein such as beta-amyloid (Ab),
wherein,
the composition comprising a PDE-5 inhibitor and an NMDA-receptor antagonist;
the PDE-5 inhibitor is selected from among mirodenafil, sildenafil, vardenafil, tadalafil, udenafil, dasantafil, avanafil; and pharmaceutically acceptable salts, solvates, hydrates, or a mixture thereof;
the NMDA-receptor antagonist is memantine, amantadine, ketamine, traxoprodil, lanicemine, rislenemdaz, pethidine, levorphanol, methadone, dextropropoxyphene, tramadol, ketobemidone, dextromethorphan (DXM), phencyclidine (PCP), and methoxetamine (MXE), pharmaceutically acceptable salts, solvates, hydrates or a mixture thereof;
the composition inhibits proinflammatory cytokines such as IL1b, IL-6, or TNFa;
the composition inhibits the growth and differentiation of nerve cells and degenerating learning and memory, to induce a decrease in intracellular Aβ, thereby increasing the protection of nerve cells and synaptic plasticity; and
the neurodegenerative disease is selected from among dementia, Parkinson's disease (PD), Dementia with Lewy body (DLB), Alzheimer's disease (AD), Huntington's disease (HD), Multiple sclerosis (MS), Vascular Dementia (VaD), Amyotrophic Lateral Sclerosis (ALS), frontotemporal dementia, or a mixed etiologies thereof.

In an embodiment of the present invention provides a composition for preventing and treating dementia comprising a phosphodiesterase 5 inhibitor and an NMDA-receptor antagonist as active ingredients.

In a certain embodiment of the present invention, the composition comprises the weight % of PDE-5 inhibitor and an NMDA-receptor antagonist is from 1:0.1 to 1:10 or 50:1, 10:1, 5:1, 2:1, 1:1, 1:2, 1:5, or 1:10.

In another embodiment, the compositions of the present invention provides a synergistic effect for:
inhibition of Aβ Oligomer/Fibril formation by reduction of Aβ aggregation;
inhibition of β-Amyloidogenic Processing decreased BACE-1;
reduction of extracellular Aβ monomers, oligomers & Aβ Fibril/Plaque by increase of the cerebral blood flow;
suppression of neuronal cell death Inhibition and promotion of neurogenesis, synaptogenesis and/or angiogenesis by activation of NO/cGMP/PKG/CREB Pathway,
restoration of synaptic plasticity (synaptic Plasticity) by activation of Wint Signaling by inhibition of DKK-1, and inhibition of production of APP and reduction of Aβ accumulation by suppression of positive feedback loop for Aβ production, and
inhibition of formation of Aβ Fibril/plaque by removal of intracellular toxic and soluble Aβ oligomers by activation of autophagy.

The phosphodiesterase 5 inhibitor of the present invention is at least one selected from among mirodenafil, sildenafil, vardenafil, tadalafil, udenafil, dasantafil, avanafil; and pharmaceutically acceptable salts, solvates, or hydrates thereof.

The pharmaceutically acceptable salts refer to a formulation of a compound that does not cause serious irritation to an organism to which the compound is administered and does not impair the biological activity and properties of the compound. The pharmaceutically acceptable salts are prepared by conventional methods well known in the art using pharmaceutically acceptable and substantially non-toxic organic and inorganic acids. The acid includes inorganic acids such as hydrochloric acid, bromic acid, sulfuric acid, nitric acid and phosphoric acid; and organic acids such as sulfonic acids, such as methanesulfonic acid, ethanesulfonic acid, and p-toluenesulfonic acid, tartaric acid, formic acid, citric acid, acetic acid, trichloroacetic acid, trifluoroacetic acid, capric acid, isobutane acid, malonic acid, succinic acid, phthalic acid, gluconic acid, benzoic acid, lactic acid, fumaric acid, maleic acid, and salicylic acid. In addition, the compound of the present invention may be reacted with a base to form ammonium salts; alkali metal salts such as sodium or potassium salts; salts such as alkali earth metal salts such as calcium or magnesium salts; salts of organic bases such as dicyclohexylamine, N-methyl-D-glucamine, tris(hydroxymethyl)methylamine; and amino acid salts such as arginine and lysine.

According to one embodiment of the present invention, examples of the pharmaceutically acceptable salts may be mirodenafil hydrochloride, sildenafil citrate, or vardenafil hydrochloride.

The hydrate refers to a compound of the present invention comprising a stoichiometric or non-stoichiometric amount of water bound by a non-covalent intermolecular force, or a salt thereof.

The solvate refers to a compound of the present invention comprising a stoichiometric or non-stoichiometric amount of a solvent bound by a non-covalent intermolecular force, or a salt thereof. Preferred solvents therefor are those that are volatile, non-toxic, and/or suitable for administration to humans.

The NMDA-receptor antagonist is memantine, amantadine, ketamine, traxoprodil, lanicemine, rislenemdaz, pethidine, levorphanol, methadone, dextropropoxyphene, tramadol, ketobemidone, dextromethorphan (DXM), phencyclidine (PCP), and methoxetamine (MXE), pharmaceutically acceptable salts, solvates, hydrates or a mixture thereof.

More preferably, the phosphodiesterase 5 inhibitor is selected from the group among mirodenafil, pharmaceutically acceptable salts, solvates, hydrates or a mixture thereof, and the NMDA-receptor antagonist is memantine, pharmaceutically acceptable salts, solvates, hydrates or a mixture thereof.

The pharmaceutical composition of the present invention may be administered orally or parenterally.

According to an embodiment of the present invention, the pharmaceutical composition of the present invention is orally administered to a subject or non-orally administered to a site other than the head. In other words, the composition of the present invention may exhibit the effect intended in the present invention even when not directly administered to the brain tissue, the body tissue surrounding the brain tissue (e.g., scalp), and a site adjacent thereto. In one specific example, the non-oral administration is subcutaneous administration, intravenous administration, intraperitoneal injection, transdermal administration, or intramuscular administration, and in another specific example, it is subcutaneous administration, intravenous administration, or intramuscular administration.

The pharmaceutically acceptable carriers comprised in the pharmaceutical composition of the present invention are those commonly used for formulation and include lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methyl hydroxybenzoate, propyl hydroxybenzoate, talc, magnesium stearate and mineral oil, but are not limited thereto. The pharmaceutical composition of the present invention may further include a lubricant, a wetting agent, a sweetening agent, a flavoring agent, an emulsifying agent, a suspending agent, and a preservative, in addition to the above components. The suitable pharmaceutically acceptable carriers and agents are described in detail in Remington's Pharmaceutical Sciences (19th ed., 1995).

The pharmaceutical composition of the present invention may be prepared in a unit dose form by formulating using a pharmaceutically acceptable carrier and/or excipient, or may be prepared by internalizing in a multi-dose container according to a method that can be easily carried out by a person skilled in the art to which the invention appertains. In this case, the formulation may be in the form of a solution, suspension, or emulsion in an oil or aqueous medium, or in the form of an extract, powder, granule, tablet, film, or capsule, and may further comprise a dispersing agent or a stabilizing agent.

In a certain embodiment, the compositions of the present invention provide synergistic effects on inhibition of proinflammatory factors to provide reduction of neuroinflammation.

In another embodiment, the compositions of the present invention provide synergistic effects on reduction of Aβ42 accumulation, to prevent and/or treat dementia through reduction of amyloid beta by combined use of an phosphodiesterase 5 (PDE5 inhibitor) and an NMDA-receptor antogonist.

### Examples

Hereafter, a more detailed description will be made using the below embodiments. However, these embodiments are only for illustrating the present invention, and the scope of the present invention is not limited by these embodiments.

### Experimental Example 1. Culture Method of IMG Cells

The IMG cells, a mouse microglia cell line used in the experiments, were cultured, and maintained in DMEM complete medium (HyClone) containing 10% fetal bovine serum (FBS; Australian Orgin, HyClone, Logan, UT, USA) and 1% penicillin/streptomycin (P/S; HyClone) at 37° C. with 5% CO₂ in a humidified CO₂ incubator (311-TIF, Thermo Fisher Scientific Forma, MA, USA). Total 2×10⁵ cells were seeded in each well of the 6-well plate and they were incubated for 24 hours in the humidified CO₂ incubator as mentioned above. Further, 100 μM of glutamate and the drugs, AR1001 and NMDA receptor antagonist (Memantine), were treated in the concentration of 2, 10, 20 μM individually or in combination.

### Experimental Example 2. RNA Extraction and cDNA Synthesis

The cells were scraped using a cell scraper, and 2 mL of the culture solution was collected in a 15 mL conical tube. It was centrifuged at 3,000 RPM for 5 minutes and the supernatant (culture solution) was discarded. The pellet was resuspended in 1 mL of Trizol, and it was transferred to 1.5 mL microfuge tube. Further, 0.2 mL of chloroform was added, and it was vortexed for 1 minutes. The microfuge tube was kept in a stand for 2 min at room temperature. After centrifugation at 12,000×g for 10 minutes at 4° C., the supernatant (approx. 500 μL) separated in a fresh microfuge tube. Equal volume of isopropanol was added to the supernatant and mixed the solution well. It was put in the microfuge tube stand for 10 minutes at room temperature and thereafter, centrifuged at 12,000×g for 10 minutes at 4° C. The supernatant was discarded, and the pellet was washed twice with 75% ethanol. The RNA pellet was dried and dissolved in 10 μL DEPC treated water. After quantification of the RNA, it was converted to cDNA following the PrimeScript™ II 1st strand cDNA Synthesis Kit (Takara) protocol.

### Experimental Example 3. Real Time RT-qPCR of Pro-Inflammatory Cytokines

The sample cDNAs were amplified with gene specific primers and SYBR green PCR master mix (ThermoFisher) in the model Quant Studio 5 Thermal cycler (Applied biosystems). The amplification conditions were as follows—polymerase activation at 50° C. for 2 minutes, predenaturation preceding at 95° C. for 10 minutes, total 40 cycles of denaturation at 95° C. for 15 seconds, annealing at 60° C. for 30 seconds and extension at 72° C. for 30 seconds. The specific primer sequences are mentioned in Table-1.

TABLE 1
Primer sequences for Real Time RT-PCR

| Gene | |
|---|---|
| name | Primer sequence |
| IL1βα | Forward 5′-AGCTTCAGGCAGGCAGTATC-3′ |
| | Reverse 5′-AAGGTCCACGGGAAAGACAC-3′ |
| TNFα | Forward 5′-AAATGGCCTCCCTCTCATCAG-3′ |
| | Reverse 5′-GTCACTCGAATTTTGAGAAGATG |
| | ATC-3′ |
| α actin | Forward 5′-CGTGCGTGACATCAAAGAGAA-3′ |
| | Reverse 5′-TGGATGCCACAGGATTCCAT-3′ |

### Experimental Example 4. Measurement of Pro-Inflammatory Cytokines Level

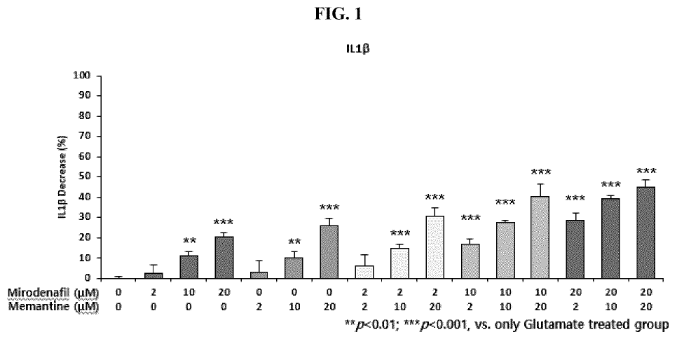

FIG. 1 shows the synergistic effect on IL-1β in the present invention of the combined treatment of mirodenafil and memantine. Here, AR1001 refers to the mirodenafil.

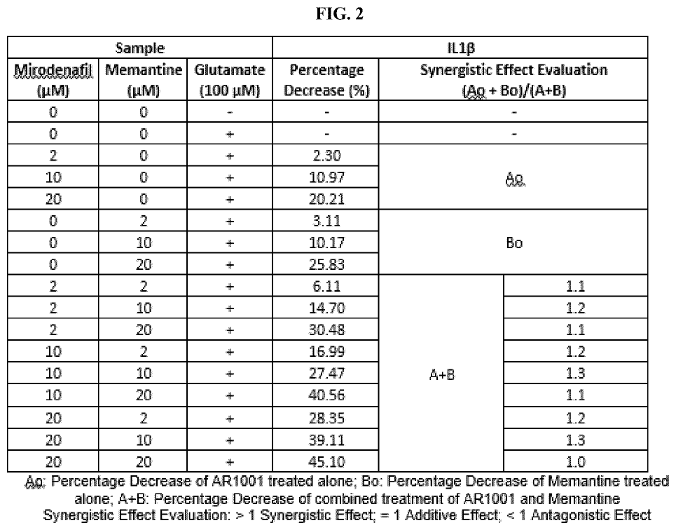

In FIG. 2, the IL-1β reduction rate for the combined treatment of 2 μM of mirodenafil and 2 μM of memantine was 6.11%; for the combination of 2 μM of mirodenafil and 10 μM of memantine, the reduction rate was 14.70%; for 2 μM of mirodenafil and 20 μM of memantine combination, the reduction rate was 30.48%; for 10 μM of mirodenafil and 2 μM of memantine combination, the reduction rate was 16.99%; for 10 μM of mirodenafil and 10 μM of memantine combination, the reduction rate was 27.47%; for 10 μM of mirodenafil and 20 μM of memantine combination, the reduction rate was 40.56%; for 20 μM of mirodenafil and 2 μM of memantine combination, the reduction rate was 28.35%; for 20 μM of mirodenafil and 10 μM of memantine combination, the IL-1β reduction rate was 39.11%; and for the combined treatment of 20 μM of mirodenafil and 20 μM of memantine, the IL-1β reduction rate was 45.10%. The reduction rates of the different combinations were significantly higher than the sum of individual reduction rates observed when treated with mirodenafil or memantine alone, which confirmed the synergistic effect beyond the additive effect.

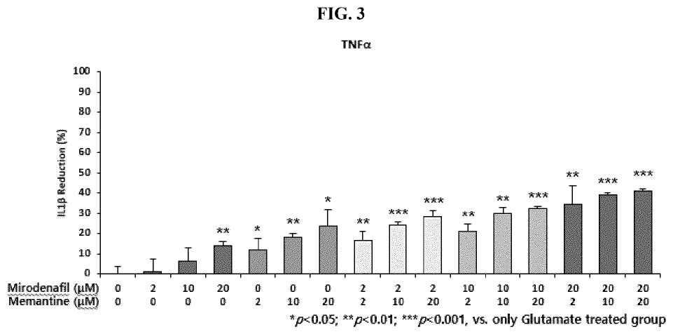

FIG. 3 shows the synergistic effect on TNF-α in the present invention of the combined treatment of mirodenafil and memantine. Here, AR1001 refers to the mirodenafil.

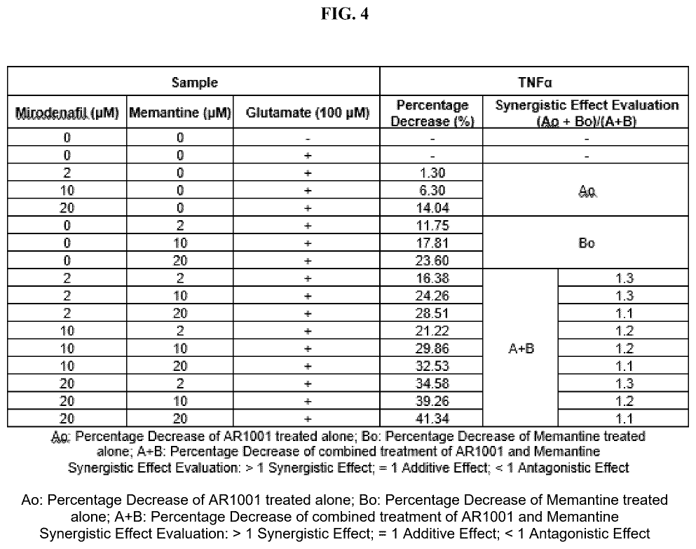

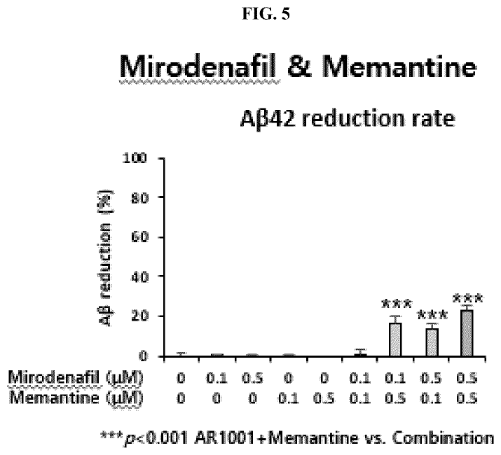

In FIG. 4, the TNF-α reduction rate for a combined treatment of 2 μM of mirodenafil and 2 μM of memantine was 16.38%; for the combination of 2 μM of mirodenafil and 10 μM of memantine, the reduction rate was 24.26%; for the combination of 2 μM of mirodenafil and 20 M of memantine, the reduction rate was 28.51%; for 10 μM of mirodenafil and 2 μM of memantine combination, the reduction rate was 21.22%; for 10 μM of mirodenafil and 10 μM of memantine combination, the reduction rate was 29.86%; for 10 μM of mirodenafil and 20 μM of memantine combination, the reduction rate was 32.53%; for 20 μM of mirodenafil and 2 μM of memantine combination, the reduction rate was 34.58%; for 20 μM of mirodenafil and 10 μM of memantine combination, the reduction rate was 39.26%; and for a combined treatment of 20 M of mirodenafil and 20 μM of memantine, the reduction rate was 41.34%. The reduction rates of the different combinations were significantly higher than the sum of individual reduction rates observed when treated with mirodenafil or memantine alone, which confirmed the synergistic effect beyond the additive effect.

### Experimental Example 5. Cell Culture

The SH-SY5Y human neuroblastoma cell line used in the experiment was purchased from American Type Culture Collection (ATCC; Manassas, VA, USA), and it was cultured in a CO₂ incubator (311-TIF, Thermo Fisher Scientific Forma, MA, USA) under the conditions of 37° C. and 5% CO2 using a DMEMIF12 Complete Medium (HyClone) containing 10% fetal bovine serum (FBS; Australian Orgin, HyClone, Logan, UT, USA) and 1% penicillin/streptomycin (P/S; HyClone).

### Experimental Example 6. Neuron-Like Differentiation of SH-SY5Y Cells Using all-Trans-Retinoic Acid (RA)

2×10⁴ cells/well were dispensed into a 96-well plate to evaluate cytotoxicity, and 2×10⁵ cells were dispensed into a T-25 flask to check neuronal cell death, neuronal inflammatory response, protein expression changes related to neurotransmitters and synaptic plasticity, and activity of acetylcholinesterase (AChE) activity. For cell fixation and stabilization, a DMEM/F12 Complete Medium (HyClone) containing 10% FBS (HyClone) and 1% P/S (HyClone) was used to culture for 24 hours in a CO₂ incubator (Thermo Fisher Scientific Forma) under the conditions of 37° C. and 5% CO₂. 24 hours after cell dispensing, the cell culture medium was removed for neuron-like differentiation and replaced with a DMEM/F12 differentiation medium containing 1% FBS (HyClone), 1% P/S (HyClone), and 10 μM all-trans-retinoic acid (RA; Sigma-Aldrich, St. Louis, MO, USA). On the third day of differentiation, the medium was replaced with a new DMEM/F12 differentiation medium. On the sixth day of differentiation, the medium for the untreated control group was replaced with a new DMEM/F12 differentiation medium, and the sample treated group was replaced by adding a new DMEM/F12 differentiation medium under various conditions.

### Experimental Example 7. Formation and Treatment of Amyloid β(Aβ)1-42

To form Aβ1-42 oligomers, human Aβ1-42 (Abcam, Cambridge, MA, USA) was added to a DMEM/F12 Complete Medium (HyClone) containing 1% FBS (HyClone) and 1% P/S (HyClone) to make 10 μM and left for three hours in a CO₂ incubator (Thermo Fisher Scientific Forma) under the conditions of 37° C. and 5% CO₂ to form Aβ1-42 oligomers.

To check the BDNF change, the existing cell culture medium was removed from the RA-differentiated SH-SY5Y neuron-like cell, and replaced with a DMEM/F12 Complete Medium (HyClone) containing Aβ1-42 oligomers (1 M), and cultured for 72 hours in a CO₂ incubator (Thermo Fisher Scientific Forma) under the conditions of 37° C. and 5% CO₂ to induce Aβ1-42 oligomer-induced cell damage.

After 72 hours, the culture medium was removed, and then the DMEM/F12 Complete Medium (HyClone) containing Aβ1-42 oligomers (1 μM or 10 M) was treated alone or in combination with mirodenafil and memantine and cultured for 24 hours in a CO₂ incubator (Thermo Fisher Scientific Forma) under the conditions of 37° C. and 5% CO₂, and then the experiment was carried out.

### Experimental Example 8. Measurement of Amyloid β (Aβ)42 by Human ELISA (Enzyme-Linked Immunosorbent Assay) Kit

To measure the amount (pg/mL) of Aβ42, 50 μL of cell culture media were placed into a 96 well plate. Further, 50 μL of Hu Aβ42 detection antibody solution was added in each well, and it was incubated for 3 hours at room temperature on orbital shaker. The solution was discarded, it was washed with 1× wash buffer, 100 μL of anti-rabbit IgG HRP antibody was added to it, and it was incubated for 30 min at room temperature. The solution was discarded entirely again, it was washed with 1× wash buffer, 100 μL of stabilized chromogen was added to it, and it was incubated for 30 min at room temperature in a dark room. Finally, 100 μL of stop solution was added to it and absorbance was measured at 450 nm within 2 hours.

### Examples 5 to 8

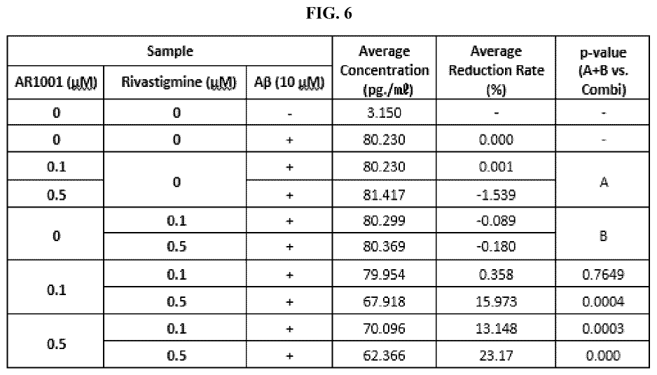

In FIG. 6, embodiment 1 is a combined treatment of 0.1 μM of mirodenafil and 0.1 μM of memantine, embodiment 2 is a combined treatment of 0.5 μM of mirodenafil and 0.1 μM of memantine, and embodiment 3 is a combined treatment of 0.5 μM of mirodenafil and 0.5 μM of memantine. For reference, AR1001 in FIG. 6 refers to mirodenafil.

### Comparative Examples 1 to 4

In FIG. 6, comparative examples 1 and 2 are treatments with 0.1 μM and 0.5 μM of mirodenafil alone, and comparative examples 3 and 4 are treatments with 0.1 μM and 0.5 μM of memantine alone.

The results indicate that the Aβ reduction rate of 15.97% in embodiment 1 for a combined treatment of 0.1 μM of mirodenafil and 0.5 μM of memantine; the Aβ reduction rate of 13.15% in embodiment 2 for a combined treatment of 0.5 μM of mirodenafil and 0.1 μM of memantine; and the Aβ reduction rate of 23.18% in embodiment 3 for a combined treatment of 0.5 μM of mirodenafil and 0.5 μM of memantine were significantly higher than the sum of the reduction rates A and B for treatment of mirodenafil or memantine alone, which confirmed that an effect beyond the additive effect can be recognized.

In other words, referring to the Aβ reduction rates of comparative examples 1 and 2 for treatments with 0.1 μM and 0.5 μM of mirodenafil alone as A1 and A2, respectively, and referring to the Aβ reduction rates of comparative examples 3 and 4 for treatments with 0.1 M and 0.5 μM of memantine alone as B1 and B2, respectively, it can be seen that the Aβ reduction rate of 15.97% in embodiment 1 is significantly higher than ‘A1+B2=−0.18%’, the Aβ reduction rate of 15.97% in embodiment 2 is significantly higher than ‘A2+B1=−1.63%’, and the Aβ reduction rate of 15.97% in embodiment 3 is significantly higher than ‘A2+B2=−1.72%’.

The present invention described above is merely illustrative and a person skilled in the art to which the present invention appertains will understand that various modifications and other equivalent embodiments are possible therefrom. Therefore, it will be understood that the present invention is not limited to the form mentioned in the detailed description above. Therefore, the true scope of the technical protection of the present invention should be determined by the technical idea of the appended scope of claims. In addition, it is to be understood that the present invention covers all modifications, equivalents, and substitutions within the spirit and scope of the present invention defined by the appended scope of claims.

### SEQUENCE LIST

| | | SEQ ID |
|---|---|---|
| Description | Sequence | NO. |
| IL1beta forward primer | 5′-AGCTTCAGGC | 1 |
| | AGGCAGTATC-3′ | |
| IL1beta reverse primer | 5′-AAGGTCCACG | 2 |
| | GGAAAGACAC-3′ | |
| TNFalpha forward primer | 5′-AAATGGCCTC | 3 |
| | CCTCTCATCAG-3′ | |
| TNFalpha reverse primer | 5′-GTCACTCGAA | 4 |
| | TTTTGAGAAGATG | |
| | ATC-3′ | |
| beta actin forward primer | 5′-CGTGCGTGACA | 5 |
| | TCAAAGAGAA-3′ | |
| beta actin reverse primer | 5′-TGGATGCCACA | 6 |
| | GGATTCCAT-3′ | |

1-16. (canceled)

17. A pharmaceutical composition comprising:
at least one phosphodiesterase 5 inhibitor; and
at least one N-methyl-D-aspartate-receptor (NMDA-receptor), or pharmaceutically acceptable salts, solvates, hydrates of said phosphodiesterase 5 inhibitor and said NMDA-receptor.

18. The pharmaceutical composition of claim 17, wherein the phosphodiesterase 5 inhibitor comprises mirodenafil, sildenafil, vardenafil, tadalafil, udenafil, dasantafil, avanafil, derivatives and mixtures thereof.

19. The pharmaceutical composition of claim 17, wherein the phosphodiesterase 5 inhibitor is mirodenafil.

20. The pharmaceutical composition of claim 17, wherein the NMDA-receptor antagonist comprises memantine, amantadine, ketamine, traxoprodil, lanicemine, rislenemdaz, pethidine, levorphanol, methadone, dextropropoxyphene, tramadol, ketobemidone, dextromethorphan (DXM), phencyclidine (PCP), or methoxetamine (MXE), and mixtures thereof.

21. A method for preventing and/or treating dementia, comprising administering an effective amount of the pharmaceutical composition of claim 17.

22. A method for preventing or treating neuroinflammation comprising:
administering an effective amount of the pharmaceutical composition of claim 17.

23. A method for preventing and/or inhibiting formation and/or accumulation of beta-amyloid comprising:
administering an effective amount of the pharmaceutical composition of claim 17.

24. A method for preventing or treating a neurodegenerative disease comprising:
administering an effective amount of the pharmaceutical composition of claim 17.

25. The method of claim 24, wherein the neurodegenerative disease is dementia, Parkinson's disease (PD), Dementia with Lewy body (DLB), Alzheimer's disease (AD), Huntington's disease (HD), Multiple sclerosis (MS), Vascular Dementia (VaD), Amyotrophic Lateral Sclerosis (ALS), frontotemporal dementia, or a mixed etiologies thereof.

26. A method for inhibiting Aβ Oligomer/Fibril formation by reducing of Aβ aggregation comprising:
administering an effective amount of the pharmaceutical composition of claim 17.

27. A method for inhibiting β-Amyloidogenic processing by reducing BACE-1 comprising:
administering an effective amount of the pharmaceutical composition of claim 17.

28. A method for reducing extracellular Aβ monomers, oligomers & Aβ Fibril/Plaque by increasing cerebral blood flow comprising:
administering an effective amount of the pharmaceutical composition of claim 17.

29. A method for suppressing neuronal cell death, promoting neurogenesis, synaptogenesis and/or angiogenesis by activating NO/cGMP/PKG/CREB Pathway comprising:
administering an effective amount of the pharmaceutical composition of claim 17.

30. A method for restoring synaptic plasticity by activating Wint Signaling by inhibiting DKK-1 comprising:
administering an effective amount of the pharmaceutical composition of claim 17.

31. A method for inhibiting production of APP and/or reducing Aβ accumulation by suppressing positive feedback loop for Aβ production comprising:
administering an effective amount of the pharmaceutical composition of claim 17.

32. A method for inhibiting formation of Aβ Fibril/plaque by removing intracellular toxic and soluble Aβ oligomers by activating autophagy comprising:
administering an effective amount of the pharmaceutical composition of claim 17.

33. A pharmaceutical composition comprising:
mirodenafil and memantine, or pharmaceutically acceptable salts, solvates, hydrates, derivatives thereof.

34. A method for preventing and/or treating dementia, comprising administering an effective amount of the pharmaceutical composition of claim 33.

35. A method for preventing or treating neuroinflammation comprising:
administering an effective amount of the pharmaceutical composition of claim 33.

36. A method for preventing and/or inhibiting formation and/or accumulation of beta-amyloid comprising:
administering an effective amount of the pharmaceutical composition of claim 33.

37. A method for preventing or treating a neurodegenerative disease comprising:
administering an effective amount of the pharmaceutical composition of claim 33.

38. The method of claim 37, wherein the neurodegenerative disease is dementia, Parkinson's disease (PD), Dementia with Lewy body (DLB), Alzheimer's disease (AD), Huntington's disease (HD), Multiple sclerosis (MS), Vascular Dementia (VaD), Amyotrophic Lateral Sclerosis (ALS), frontotemporal dementia, or a mixed etiologies thereof.

39. A method for inhibiting Aβ Oligomer/Fibril formation by reducing Aβ aggregation comprising:
administering an effective amount of the pharmaceutical composition of claim 33.

40. A method for inhibiting β-Amyloidogenic processing by reducing BACE-1 comprising:
administering an effective amount of the pharmaceutical composition of claim 33.

41. A method for reducing extracellular Aβ monomers, oligomers & Aβ Fibril/Plaque by increasing the cerebral blood flow comprising:
administering an effective amount of the pharmaceutical composition of claim 33.

42. A method for suppressing neuronal cell death, promoting neurogenesis, synaptogenesis and/or angiogenesis by activating NO/cGMP/PKG/CREB Pathway comprising:
administering an effective amount of the pharmaceutical composition of claim 33.

43. A method for restoring synaptic plasticity by activating Wint Signaling and/or inhibiting DKK-1 comprising:
administering an effective amount of the pharmaceutical composition of claim 33.

44. A method for inhibiting production of APP and/or reducing Aβ accumulation by suppressing positive feedback loop for Aβ production comprising:
administering an effective amount of the pharmaceutical composition of claim 33.

45. A method for inhibiting formation of Aβ Fibril/plaque by removing intracellular toxic and soluble Aβ oligomers by activating autophagy comprising:
administering an effective amount of the pharmaceutical composition of claim 33.
